(19) United States
(12) Patent Application Publication
(10) Pub. No.: US 2003/0161795 A1
(43) Pub. Date: Aug. 28, 2003

(54) Substantially visibly transparent topical physical sunscreen formulation

(76) Inventors: Takuya Tsuzuki, Kalamunda (AU); Malcolm Nearn, Kentlyn (AU); Geoff James Trotter, Yokine (AU)

Correspondence Address: John C. Kerins, MILES & STOCKBRIDGE P.C., Suite 500, 1751 Pinnacle Drive, McLean, VA, 22102-3833, US

(21) Appl. No.: 10/324,112

(22) Filed: Dec. 20, 2002

(30) Foreign Application Priority Data
Feb. 27, 2002 (AU) PS 0808
Sep. 23, 2002 (AU) 2002951590

(51) Int. Cl. A61K 7/42

(52) U.S. Cl. 424/59

(57) ABSTRACT

A topically applied sunscreen composition is provided, which by use of nano-sized particles of a physical UV screening agent in a dermatologically acceptable carrier, provides a dermatologically acceptable level of SPF and broad spectrum protection from UVA and UVB radiation, without the need to include chemical UV screening agents in the composition.

### FIELD OF THE INVENTION

This invention relates to a substantially visibly clear and transparent topical sunscreen composition having a dermatologically acceptable level of SPF and broad spectrum UVA/UVB protection for shielding the skin from ultraviolet radiation relying on physical UV screening agents alone. The composition comprises a sufficient weight percentage of nano-sized particles of a physical UV screening agent to provide the desired level of SPF without any chemical UV screening agents being added.

### BACKGROUND TO THE INVENTION

It is well established that UV radiation with wavelengths between 290 nm and 400 nm damages the human epidermis, both in the short term, leading to sunburn, and, in the long term, leading to premature aging of the skin and skin cancer. UVB radiation, having wavelengths between 290 and 320 nm, is well known to cause bums and erythema and should be screened out. UVA contributes to the damage caused by UVB, and in addition may cause other harmful effects, such as polymorphic light eruption and photosensitivities to certain chemicals.

Sunscreen compositions are broadly classified into “chemical” (organic) or “physical” (inorganic) sunscreens depending on the nature of the active ingredient which acts to screen out UVA and UVB radiation. Chemical sunscreens typically contain conjugated molecular structures that absorb UVB and/or UVA wavelengths and then retransmit the energy at longer safer wavelengths. Usually the range of wavelengths against which chemical sunscreens protect is narrower than for the physical sunscreens and only partial protection is achieved against UVA, even in what are labeled “broad spectrum products”. Physical sunscreens on the other hand, typically consist of a dispersion of particles of inert inorganic compounds which preferentially absorb UV radiation and which may also scatter UV and visible radiation depending on the size of the particles, the wavelength of the UV radiation, and the difference in refractive index of the dispersed particles and the dispersion medium. It is well known in the cosmetics industry that certain metal oxides, including zinc oxide and titanium oxide, are effective physical UV screening agents. Zinc oxide in particular is known to have a high absorptance to UV radiation over virtually the entire spectrum of UVB and UVA radiation whereas titanium dioxide provides UV protection over a more limited spectrum. The inclusion of zinc oxide as a physical UV absorber in sunscreens is known.

Physical sunscreens are preferred over chemical sunscreens in that chemical sunscreens are known to be photosensitive and may be degraded or altered by UV radiation. Moreover, the long-term effects of chemical sunscreens on skin and general health of the user are unknown. Physical sunscreens are preferable, particularly those containing zinc oxide, as such physical sunscreens are known to be UV stable and exhibit no known adverse effects associated with long-term contact with the skin.

The major limiting factor in the use of physical UV screening agents is the tendency for sunscreen formulations including such physical UV screening agents to appear white on the skin due to excessive scattering of light from the particles contained within such sunscreen formulations. This results in low cosmetic acceptability and marketability of sunscreen formulations which rely on physical UV screening agents alone.

The efficacy of sunscreens is usually characterised by an SPF (Sun Protection Factor) which is a measure of the increase in exposure time to UV radiation required to induce erythema. The SPF is typically expressed as a number followed by a “+”. For example, an SPF of 15+ indicates that the SPF is at least 15. Dermatologically acceptable levels of SPF vary from country to country. In Australia sunscreen formulations have an SPF of 15+or 30+. SPF tests are conducted “in-vivo” or “in-vitro”. In Australia, in-vivo SPF tests are carried out according to Australian Standard AS/NZS 2604:1998.

Since sunburn is mostly associated with UVB radiation, the commonly used SPF tests measure protection against UVB radiation rather than UVA. In particular, the UV emission of the solar simulator used in the SPF test may be deficient in UVA radiation above about 350 nm, when compared with the spectrum of natural sunlight. This may be important because there is mounting evidence that exposure to UVA may be a significant risk factor for premature aging of the skin and certain forms of skin cancer. Furthermore various short-term and long-term adverse effects may be relatively more sensitive to UVA than is sunburn erythema.

In view of growing concerns regarding the effect of UVA radiation, several additional tests have been proposed which measure the ability of the sunscreen to block out radiation over the entire UV spectrum. Of particular relevance is the UVA/UVB ratio which is equal to the ratio of the UVA to UVB radiation absorbed by the sunscreen. A further parameter used to evaluate the effectiveness of sunscreens over the entire UV spectrum is the critical wavelength parameter, defined as the wavelength above which 90% of the total UV radiation is absorbed.

The results of any of the above-mentioned tests are dependent on the particular thickness of the layer of the sunscreen composition or formulation being tested. Most SPF tests require that 2 mg/cm² of the sunscreen composition or formulation, corresponding to a layer thickness of about 20 microns, to be applied to the subject. If a thinner layer of sunscreen is used, the degree of UV blockage is lowered.

In sunscreens containing physical sunscreens the transparency decreases with increasing concentration of the physical sunscreen particles because of increased scattering of light by the particles, which causes a whitening effect in the layer of sunscreen. Thus, for a given layer thickness there is typically a trade-off between the transparency and whiteness of the layer and the concentration of physical screening in the layer. In known commercially available sunscreens the whitening effect limits the maximum concentration of physical UV screening agents, such as zinc oxide or titanium oxide, in sunscreens to values which are unable to provide adequate UVA/UVB protection. As a consequence, acceptable values of SPF can only be achieved by adding chemical UV screening agents to the sunscreen.

As mentioned above, one of the main limitations of the use of physical UV screening agents in sunscreens is the problem of whiteness left on the skin after the sunscreen has been applied. If an image-conscious user of the sunscreen applies a thin layer of that sunscreen to avoid this whiteness effect, the effective SPF will be less than that measured in the standard tests due to the fact that any SPF rating is dependent on the thickness of the layer of sunscreen tested. Thus the SPF measured in an SPF test may not be obtained by the user in the actual usage of the product if they are concerned about avoiding whitening.

There is therefore a need for an improved topical sunscreen formulation for shielding the skin from ultraviolet radiation to reap the benefit of the ability of physical sunscreens to effectively block out UVA and UVB radiation while avoiding the photo reactivity problem of chemical sunscreens. The full SPF rating of such a sunscreen can be exploited without a corresponding reduction in the cosmetic desirability of the sunscreen product.

There is an existing trend in the sunscreen industry to develop and use sunscreen formulations containing zinc oxide of smaller and smaller particle size to reduce the whiteness and improve the transparency of sunscreen formulations. U.S. Pat. No. 5,573,753 for example, discloses a method of preparing sunscreens containing zinc oxide particles of 5 nm to 150 nm which is claimed to be substantially transparent to visible light while screening UV radiation. U.S. Pat. No. 5,531,985 describes a sunscreen which includes a dispersion of zinc oxide particle 10 nm to 100 microns in size.

Neither of these patents make mention of the SPF rating of such a composition, the thickness of the layer of such a sunscreen for which substantial visible transparency is achieved, nor the weight percentage of zinc oxide which may be included in such a sunscreen and still provide “substantial visible transparency”. If applied thinly enough or if loaded with only a small percentage of zinc oxide particles, any sunscreen composition would be able to claim to be transparent. However such a sunscreen would not have a dermatologically acceptable level of SPF.

Claims to a substantially visibly transparent sunscreen including zinc oxide particles have been made before. For example, U.S. Pat. No. 5,587,148 (Mitchell) is directed towards a sunscreen including substantially pure micronised particles of zinc oxide of a specific average particle size range less than about 0.2 microns with a particular purity. Mitchell claimed that when such particles were mixed with a dermatologically acceptable liquid carrier, these particles become substantially uniformly dispersed and shielded the skin from both UVA and UVB solar radiation which remaining substantially visibly transparent. However commercially available sunscreens incorporating the zinc oxide particles of Mitchell have all needed to rely on the inclusion of chemical UV blockers to achieve the visible transparency whilst maintaining the requisite SPF. Tests have shown that a sunscreen relying on the zinc oxide powders of Mitchell alone would have resulted in a sunscreen with poor transparency and significant whiteness at acceptable levels of SPF. For example, UV-visible transmittance data taken from the “Affidavit under 37 C.F.R. 1,132 (Exhibit A)” filed with the USPTO during re-examination of U.S. Pat. No. 5,587,148 (available from the USPTO) indicated a total transmittance of only 18% at a wavelength of 550 nm corresponding to the mid point of the visible spectrum.

There are at present no commercially available sunscreens which are visibly clear and transparent on the skin that rely solely on the use of physical sunscreens.

It is noted that much of the prior art in this area is characterised by a lack of quantitative assessment of transparency and whiteness, despite the fact that precise scientifically acceptable definitions and measurement techniques exist. Furthermore, it is noted that transparency and whiteness of a sunscreen layer depend directly on the thickness of the layer and without explicit knowledge of the layer thickness, the values of transparency and whiteness have no meaning.

An object of the present invention is to provide a substantially visibly transparent topical sunscreen composition which for the first time is able to provide the requisite level of SPF without the need to include photodegrading and potentially biosensitive UV screening agents.

Throughout this specification the term “transparent” is to be understood as meaning the “property of transmitting rays of light through its substance so that bodies situated beyond or behind can be distinctly seen (as distinguished from translucent and opposed to The term “dermatologically acceptable level of SPF” has been chosen to cover the situation of various countries setting a minimum SPF that a given sunscreen must comply with in order to be able to be sold to consumers in a given country. For example, based on current regulations, many South East Asian countries only require that sunscreen products have an SPF of 8+. In Australia, the majority of sunscreens sold have a minimum SPF of 15+.

For any given jurisdiction a sunscreen formulator would be readily able to determine the weight percentage of the physical UV screening agent required to achieve the requisite level of SPF.

Generally the sunscreen composition would rely on zinc oxide alone as the physical UV screening agent and the majority of the testing included in the following description relates to the use of zinc oxide alone. However it is within the scope of the present invention for titanium dioxide, cerium oxide or other physical UV screening agents or mixtures thereof to be included along with zinc oxide in the role of the physical UV screening agent to achieve the desired level of SPF. Zinc oxide is preferred due to its superior performance as a UV screening agent over a broader range of UV radiation. It is to be clearly understood that the sunscreen composition of the present invention would still achieve the promise of claim 1 with up to 10% of titanium dioxide, or other physical UV screening agents or mixtures thereof, used in addition with zinc oxide as the physical UV screening agent.

Preferably said substantially visibly clear and transparent sunscreen composition has a specular extinction coefficient of less than 2 (wt % mm)⁻¹ measured at a wavelength of 550 nm. More preferably still, said substantially transparent dispersion has a specular extinction coefficient of less than 1 (wt % mm)⁻¹ measured at a wavelength of 550 nm.

The value of the specular extinction coefficient provides a unique measure of the degree of “clearness” or “lack of whiteness” achieved using the present invention. This measure is independent on the thickness of the layer of the composition being tested or applied. opaque) and the term “clear” is to be understood to mean “free from whiteness or cloudiness”.

The terms “sunscreen” and “UV screening agents” throughout this specification in no way imply or suggest that 100% blockage of UV radiation occurs. These terms are merely used to describe the role of the agent or composition in reducing the extent to which UV radiation is able to access the skin of the user.

It will be clearly understood that, although a number of prior art publications are referred to herein, this reference does not constitute an admission that any of these documents forms part of the common general knowledge in the art, in Australia or in any other country.

Throughout this specification the term “comprising” is used inclusively, in the sense that there may be other features and/or steps included in the invention not expressly defined or comprehended in the features or steps subsequently defined or described. What such other features and/or steps may include will be apparent from the specification read as a whole.

### SUMMARY OF THE INVENTION

According to one aspect of the present invention, there is provided a substantially visibly clear and transparent topical sunscreen composition having a dermatologically acceptable level of SPF and broad spectrum UVA/UVB protection for shielding the skin from ultraviolet radiation, said composition comprising:

a sufficient weight percentage of nano-sized particles of a physical UV screening agent to provide said dermatologically acceptable level of SPF in a dermatologically acceptable carrier whereby said composition contains no chemical UV screening agents.

The term “composition” is intended to cover a dispersion, an emulsion (either a cream or a lotion), a stick, a gel, a spray, a clear lotion, or a wipe or any other composition suitable for use in protecting skin against sun damage. The dispersion or emulsion may be a water-in-oil emulsion, or an oil-in water emulsion, or a multiple phase emulsion.

It is envisaged that an amount of one or more chemical UV screening agents may be added to the sunscreen composition of the present invention as an alternative to the physical UV screening agent. However it is to be understood that the dermatologically acceptable SPF is achievable without the need for any chemical UV screening agents to be added and the addition of chemical UV screening agents is not preferred.

Preferably, the nano-sized zinc oxide particles are substantially fully dispersed, have a mean particle size of less than 30 nm and have a narrow particle size distribution characterised in that, based on a number-weighted size distribution measured by photo-correlation spectroscopy, the number-weighted size distribution has a standard deviation of less than 20 nm. More preferably, the number-weighted size distribution measured by photo-correlation spectroscopy has a standard deviation of less than 10 nm. More preferably still, the number-weighted size distribution measured by photo-correlation spectroscopy has a standard deviation of less than 5 nm.

Preferably said particles have a photoactivity which is reduced by treatment with a surfactant.

Preferably said surfactant is a steric surfactant. The steric surfactant could be chosen from the list of stearic acid, recinolieic acid, poly 12-hydroxy stearic acid, metal hydroxy stearic acid, oleic, palmitic, lauric, plearagonic and myristic acids and esters of those acids (or connotations thereof), as well as polyelectrolytes such as sodium polyphosphate. Alternatively said powder may be coated with a layer of one or more of a metal hydroxide, a metal oxide or a hydrous metal oxide. A wide range of metals are considered suitable but the preferred metals are silicon, aluminium, zirconium.

### BRIEF DESCRIPTION OF THE DRAWINGS

In order to facilitate a more detailed understanding of the nature of the invention preferred embodiments will now be described in detail, by way of example only, with reference to the accompanying drawings, in which:

FIG. 1 illustrates graphically the particle size distribution of a ZnO powder suitable for use in a topical sunscreen composition according to at least one embodiment of the present invention as measured by Photon Correlation Spectroscopy;

FIG. 2 illustrates a Transmission Electron Micrograph of particles of a ZnO powder suitable for use in a topical sunscreen composition according to at least one embodiment of the present invention;

FIG. 3 illustrates graphically the UV-Vis specular transmittance spectra of 0.1 wt % slurry of the ZnO powder of Example 1 in deionised water;

FIG. 4 illustrates graphically the effect of the size of the ZnO particles on the UV-Vis specular transmittance spectra;

FIG. 5 illustrates graphically the UV-Vis specular transmittance spectra of 0.01 wt % ZnO for the samples of Example 2 dispersed in deionised water;

FIG. 6 illustrates graphically the decay rate of the indicators for samples A, B and C of Example 3 under UV exposure;

FIG. 7 illustrates graphically the UV-Vis specular transmittance spectra of ZnO of Example 4 dispersed into Isostearyl Benzoate from aqueous solution;

FIG. 8 illustrates graphically the UV-Vis specular transmittance spectra of ZnO of Example 5 dispersed into canola oil from aqueous solution;

FIG. 9 illustrates graphically the UV-Vis specular transmittance spectra of ZnO of Example 6 dispersed into hexane from aqueous solution, (a) as dispersed, (b) after drying and redispersion;

FIG. 10 illustrates graphically the effect of mean particle size of a preferred embodiment of the ZnO powder according to Example 7 of the present invention on UV-Vis total transmittance measurements;

FIG. 11 illustrates graphically the effect of mean particle size of a preferred embodiment of the ZnO powder according to Example 7 on % total transmittance at 550 nm;

FIG. 12 illustrates graphically the effect of mean particle size of a preferred embodiment of the ZnO powder according to Example 7 on % total transmittance at 330 nm;

FIG. 13 illustrates graphically the effect of mean particle size of a preferred embodiment of the ZnO powder according to Example 7 on the Whiteness index for 20 micron thick films;

FIG. 14 illustrates graphically the effect of particle size and of a preferred embodiment of the ZnO powder according to Example 7 on in-vitro SPF (Transpore tape method);

FIG. 15 illustrates graphically the absorptance spectra for sample consisting of 30% of a preferred embodiment of the ZnO powder according to Example 7 dispersed in Isostearyl Benzoate;

FIG. 16 illustrates graphically the effect of mean particle size on the specular transmittance at 550 nm for various values of in-vitro SPF.

FIG. 17 illustrates graphically an observed linear correlation between the in-vitro SPF and in-vivo SPF values;

FIG. 18 illustrates graphically the data of FIG. 23 redrawn using the correlation of FIG. 24 showing the effect of mean particle size on the specular transmittance at 550 nm for various values of in-vivo SPF;

FIG. 19 illustrates graphically the extinction coefficient as a function of the particle size for a specular transmittance of 550 μm using the data of FIG. 11;

FIG. 20 illustrates graphically the UVVis specular transmittance spectra of 16 wt % ZnO in chemical-free sunscreen formulations;

FIG. 21 illustrates graphically the specular extinction coefficient of 16 wt % ZnO in chemical-free sunscreen formulations;

FIG. 22 illustrates graphically the CIE L* coordinate of 16 wt % ZnO in chemical-free sunscreen formulations for 20 micron thick films;

FIG. 23 illustrates images of 8 micron thick films of 16 wt % ZnO for various chemical-free sunscreen formulations;

FIG. 24 illustrates graphically In-vitro SPF levels calculated from total transmittance data of sunscreen formulations using a quartz cell having an optical-path-length of 8 micron;

FIG. 25 illustrates graphically the relationship between in-vitro SPF measured using 8 micron cell and in-vivo SPF values, for 25 nm sized ZnO nanoparticle suspension in Finsolv-TN having different concentrations;

FIG. 26 illustrates graphically In-vivo SPF levels for the sunscreen formulations of FIGS. 20 and 21;

FIG. 27 illustrates graphically the extinction coefficient for a specular transmittance at 550 nm for the 25 nm ZnO for the sunscreen formulations of Examples 7, 8 and 9);

FIG. 28 illustrates graphically the UVVis total transmittance spectra of Samples 1 and 2 of Example 12 for ZnO suspensions in oil phases; and,

FIG. 29 illustrates graphically a comparison of the whiteness index of a sunscreen composition of the present invention with the whiteness index of various other commercially available products as a function of the weight percentage of zinc oxide particles included in that composition for 20 micron thick films.

### DESCRIPTION OF THE PREFERRED EMBODIMENTS

The present invention derives from the ability of the applicant to be able to manufacture nano-sized zinc oxide particles with a far greater control on particle size, size distribution and agglomeration than previously achievable. Having manufactured such particles, the applicant realised that when formulated into a sunscreen, the particles exhibited unexpectedly high transmittance in the visible spectrum and far less whiteness than any other commercially available zinc oxide particles used in such sunscreens.

It was then realised that for the first time it was possible to add a sufficient quantity of such particles to a sunscreen composition or formulation and achieve dermatologically acceptable levels of SPF using the zinc oxide particles alone. The amount of zinc oxide particles required to be included in any given sunscreen formulation is directly dependent on the level of SPF required for that formulation and the particular ingredients selected by the formulator. Generally speaking, a higher weight percentage of zinc oxide particles will need to be included to provide the formulation with an SPF of 30+than would be required to provide an SPF of 8+.

The weight percentage of zinc oxide required to provide a particular level of SPF is dependent on the other ingredients included in a given formulation. For a sunscreen formulation relying on the nano-sized zinc oxide particles of the preferred embodiments of the present invention, typically 5-12 wt % may be required to achieve an SPF of 15+, and at least 12 wt % to achieve an SPF of 30+.

Again it is stressed that the specific amount of zinc oxide included in a formulation will vary depending on the particular ingredients selected by a formulator and such persons would as a matter of routine, adjust the amount of zinc oxide particles added to the formulation to achieve the level of SPF required.

Whilst the sunscreen formulation of the preferred embodiments of the present invention does not need to rely on the inclusion of one or more chemical UV screening agents, the amount of zinc oxide particles required to be included in a given formulation to achieve the required dermatologically acceptable level of SPF may be reduced by chemical UV screening agents added to such a formulation. Again it is considered that it would be a matter of routine experimentation for a sunscreen formulator to determine the specific range of zinc oxide required to be added to achieve the required level of SPF when chemical UV screening agents are included.

The zinc oxide particles of the present invention have a mean particle size of less than 30 nm. In addition to the small mean particle size the size distribution of such particles is preferably very narrow. The manufacturing techniques of the present invention allow for the size distribution to be controlled in such a way that based on a number-weighted size distribution measured by photo-correlation spectroscopy, the number-weighted size distribution has a standard deviation of less than 20 nm. More preferably, the number-weighted size distribution has a standard deviation of less than 10 nm and more preferably still, less than 5 nm.

It is believed that the combination of low mean particle size and narrow size distribution provide the observed increase in transparency and absorption of UVA and UVB radiation. Without wishing to be bound by theory, it is believed that the increase in visible transmittance is due to the absence of relatively large particles which may contribute to excess scattering of visible light.

It has been found that keeping agglomeration to a minimum is very important in keeping the effective mean particle size of the particles within the desired range. Conventional wisdom in the art of sunscreen formulation teaches that particles with such a small mean particle size should be avoided as such particles are considered to be extremely difficult to disperse and thus not particularly suitable for inclusion in a composition intended for use as a sunscreen. Surprisingly it is found that the particles manufactured according to the process described herein are easy to disperse in a wide range of carriers using standard dispersion equipment and techniques.

The manufacturing process developed by the applicant has resulted in the production of zinc oxide particles that have a relatively non-reactive surface compared with other conventional manufacturing methods. Moreover the surface may be treated after manufacture to further assist in minimising agglomeration. For example, stearic acid coatings may be used to improve dispersibility. Alternatively, the particles may be coated with one or more layers consisting of hydroxides, oxides or hydrous oxides of silicon, aluminium, zirconium or other suitable metal or a mixture thereof.

Zinc oxide particles used in accordance with the present invention may be manufactured using the mechano-chemical process described in the applicant's U.S. Pat. No. 6,203,768, the contents of which are incorporated herein by reference. Mechano-chemical processing involves a mechanically activated chemical reaction between a precursor metal compound and a suitable reactant during mechanical milling or during subsequent heat treatment of the milled powder. During mechanical activation a nano-composite structure is formed which consists of nano-sized grains of the nanophase substance within a matrix of a non-reactant diluent. The volume fraction of the diluent must be above a critical value to ensure substantially complete separation of the particles of the desired phase. Proper removal of the diluent yields substantially unagglomerated nanometre sized particles of the desired phase.

Through careful choice of the reaction chemistry, milling process and processing conditions, mechano-chemical processing can be used to economically manufacture zinc scope of the present invention. During milling, a nano-composite grain structure is formed. If the milling temperature is sufficiently high (T>140° C.), the ZnCl₂ may react with Na₂CO₃ during milling, forming nano grains of ZnO within a matrix of NaCl, with CO₂ gas being given off during the reaction. Alternatively, the milled nano-composite may be heat-treated after milling. In this case, the zinc chloride reacts with sodium carbonate during milling to form nano composite particles of zinc carbonate within the sodium chloride phase. After milling, the ZnCO₃ is converted to ZnO by heat treating the milled powder at temperatures above 250° C.

An excess non-reactant diluent phase such as NaCl may be added to promote separation of the nano-composite particles during their formation. The presence of a sufficient volume fraction of a non-reactant diluent enables separation of the zinc oxide particles and thus a minimum of agglomeration or sintering together of particles occurring during heat treatment. The volume fraction of the diluent phase should be at least 80% to ensure fully separated particles. Following heat treatment, the non-reactant diluent phase is removed by, for example, dissolution in a solvent and filtering.

It has been found that heat treating the milled powder at a temperature around 350° C. as described in Example 2 leads to a slight increase in the mean particle size with a surprising increase in the visible transparency and a decrease in the UV transparency for a sunscreen composition including such particles compared with a sample heat treated at 250° C. with a smaller mean particle size. Without wishing to be bound by theory, it is believed that this result can be attributed to the particles heat treated at 250° C. having a higher reactivity, causing an increase in the effective particle size due to agglomeration. Particles heat-treated at 350° C. on the other hand exhibited a significantly increased dispersibility which may be attribute to the higher heat treatment temperature stabilising the particle surfaces.

After removal of the non-reactant diluent, it is preferable to treat the powder particles with a steric surfactant to minimise agglomeration. Suitable surfactants include: stearic acid, recinolieic acid, poly 12-hydroxy stearic acid, metal hydroxy stearic acid, oleic acid, oxide nano powders in accordance with the present invention, the powders having not only with a smaller mean size but, equally importantly, a narrow size distribution, and enhanced stability. When the powders are dispersed into a sunscreen formulation the result is both enhanced visible transmittance and enhanced UV absorptance.

To achieve a narrow size distribution using mechano-chemical processing it is highly preferable that the milling process be designed so that it is as uniform as possible, both temporally and spatially, while still providing sufficient collision energy to mechanically activate the reactants. To ensure uniform milling of the charge during dry milling, batch milling is employed so that each particle experiences the same milling time. With the batch milling of dry constituents there is a tendency for the powder to not circulate efficiently through the mill, but rather remain near the container walls, in a zone of reduced collision energy, resulting in non-uniform, inefficient milling.

An attrition mill has been found to be a suitable mill for mechanical activation which can be scaled up for commercial production. A conventional attrition mill consists of a stationary cylindrical container filled with grinding balls that are stirred by impeller arms extending from a central drive shaft. In conventional attrition mills the impellers do not extend to the wall of the container, instead a gap equal to 3-4 ball diameters separates the ends of the impellers from the wall of the vessel to minimise wear of the container walls.

It has been found that to achieve sufficiently uniform milling conditions, it is necessary to eliminate the dead zone at the container wall by extending the impellers to the wall of the vessel. Surprisingly, it has been found that extension of the impellers has only a minor effect on wear, but, more importantly, significantly increases the efficiency and uniformity of the milling process and, as a result, a narrow particle size distribution in the resulting powder as well as a reduction in milling time.

Mechano-chemical processing to produce nano-sized zinc oxide particles is best accomplished through the milling of a precursor zinc compound such as zinc chloride and a reactant such as sodium carbonate as described in Example 2. It is however to be clearly understood that other suitable reactants may be employed and still fall within the palmitic acid, lauric acid, plearagonic acid and myristic acid or esters of these acids either alone or in combination.

The topical sunscreen composition of the present invention may be formulated by including one or more of the following components in addition to a suitable quantity of zinc oxide particles:

(a) A suitable surfactant for the zinc oxide

(b) Optionally one or more emulsifiers

(c) Optionally one or more waxes

(d) Optionally one or more electrolytes

(e) Optionally one or more dihydric or polyhydric alcohols

(f) Optionally one or more moisturisers

(g) Optionally water

(h) Optionally one or more water-soluble polymers

(i) Optionally one or more film-formers

(j) Optionally one or more water-proofing materials

(k) Optionally one or more thickeners for the oil phase

(l) Optionally one or more antimicrobial preservatives

(m) Optionally acid or alkali added to adjust the pH of the aqueous phase to above about 7.0.

(n) Optionally one or more emollients

(o) Optionally one or more antioxidants

(p) Optionally one or more free radical scavengers

(q) Optionally one or more fragrance materials

(r) Optionally one or more organic sunscreen actives

(s) Optionally one or more inorganic sunscreen actives

(t) Optionally one or more solvents for the organic sunscreens

(u) Optionally one or more materials to photostabilise the organic sunscreens

(v) Optionally one or more vitamins

(w) Optionally one or more materials to prevent or reverse the effects of premature aging of the skin by the sun.

HLB emulsifiers. Many examples of such emulsifiers are listed in McCutcheon's “Emulsifiers and Detergents.”

Waxes that may be used include, but are not restricted to one or more of the following: Ozokerite, paraffin wax, beeswax, camauba wax, ceresin, candelilla wax, castor wax, long chain fatty alcohols such as cetyl alcohol, stearyl alcohol, behenyl alcohols, and synthetic spermaceti wax.

Electrolytes that may be used include, but are not restricted to one or more of the following: salts of monovalent metals such as sodium chloride, salts of divalent metals such as magnesium sulfate.

Dihydric or polyhydric alcohols that may be used include, but are not restricted to one or more of the following: propylene glycol, sorbitol, and glycerol.

Moisturisers and skin conditioners that may be used include, but are not restricted to one or more of the following: urea, glycolic acid and its salts, lactic acid and its salts, aloe vera, sorbitol, glycerol, butylene glycol, hexylene glycol and other polyhydric alcohols, polyethylene glycol, sugar and its derivatives, starch and its derivatives, hyaluronic acid and its salts, urea, guanidine, and mixtures thereof.

Water-soluble polymers that may be used include, but are not restricted to one or more of the following: xanthan gum, cellulose derivatives, polymers of acrylic acid and derivatives, carbomers, PVP, alginates, guar gum. Other thickeners and stabilisers for the water phase may include, but are not restricted to one or more of the following: magnesium aluminium silicate, sodium aluminium silicate, colloidal silica, fumed silica, sodium stearate, acrylates/steareth 20 methacrylate copolymer (Aculyn 22), acrylates copolymer emulsion (Aculyn 33A), PEG150/decyl alcohol/SMDI copolymer (Aculyn44), PEG150/stearyl alcohol/SMDI copolymer (Aculyn46). Preferably said thickeners for the oil phase include polyethylene, hydrophobic silica, metal stearates such as zinc stearate, and any of one or more of the aforementioned waxes.

(x) Optionally one or more materials to impart a tan to the skin.

(y) Optionally volatile materials that accelerate the drying of the sunscreen product when applied to the skin

(z) Other materials of secondary benefit that are known to people familiar with the art.

While the applicant has developed for the first time a substantially visibly transparent topical sunscreen composition that need not rely on the inclusion of chemical UV screening agents to deliver a dermatologically effective level of SPF, it is within the scope of the present invention for chemical UV screening agents to be included as one of the components of the topical sunscreen composition if such components are considered more cost-effective. While product price considerations may dictate that chemical UV screening agents be included in the composition, the inclusion of such chemical UV screening agents is in no way essential to the ability of the visibly transparent topical sunscreen composition of the present invention to provide a dermatologically effective level of SPF alone.

For water-in-oil emulsions examples of suitable emulsifiers include, but are not limited to, the following: Ethoxylated sorbitan esters (available commercially under the trade name Tween); Polyethoxylated esters of hydrogenated castor oil (available commercially under the trade name Arlacel 989); Sorbitan sesquioleates (available commercially under the trade name Arlacel 83); PEG 30 Dipolyhydroxystearate (available commercially under the trade name Arlacel P135); Glycerol sorbitan oleostearate (available commercially under the trade name Arlacel 481); Polyoxyethylene Glycerol sorbitan isostearate (available commercially under the trade name Arlacel 582); PPG PEG Glycerol sorbitan hydroxyisostearate (available commercially under the trade name Arlacel 780); Glycerol sorbitan fatty acid ester (available commercially under the trade name Arlacel 986); Abil WE09; Abil Wax 9801; Monomuls 90-018 and/or Dehymuls PGPH.

Suitable emulsifiers for oil-n-water emulsions usually have HLBs (hydrophile/lipophile balances) greater than about 7. They are often used in combination with one or more low Film formers and waterproofing agents include, but are not restricted to one or more of the following: Acrylates/t-octylpropenamide copolymer (Dermacryl 79); alkylated polyvinylpyrrolidones (Antaron V216 and Antaron V220); tricontanyl polyvinylpyrrolidone (Antaron WP660).

Emollients that may be used include, but are not restricted to one or more of the following: hydrocarbon oils, such as paraffin oil or mineral oils; vegetable oils such as sunflower oil, apricot oil, jojoba oil and its derivatives, shea butter; silicone oil and its derivatives; fatty acid esters, such as isopropyl palmitate, isopropyl myristate, isopropyl neopentanoate, cetearyl octanoate, C12-15 alkyl benzoate, cetyl palmitate, octyl palmitate and mixtures thereof, silicone oils and derivatives of silicone oils.

Sunscreen compounds that may be used include, but are not restricted to one or more of the following: 2-ethylhexyl-p-methoxycinnamate, isoamyl-p-methoxycinnamate, 2-ethoxyethyl p-methoxycinnamate, 2-ethylhexyl N,N-dimethyl-p-aminobenzoate, 4-aminobenzoic acid, 2-phenyl-benzimidazole-5-sulfonic acid and its potassium sodium and triethanolamine salts, homosalate, oxybenzone, 2-ethylhexyl salicylate, 3-(4′-methylbenzylidene)d-1-camphor, Benzophenone-2, Benzophenone-4, Benzophenone-5, Dioxybenzone, menthyl anthranilate, octocrylene, octyl triazone, triethanolamine salicylate, titanium dioxide, PEG25 PABA, avobenzone and mixtures thereof.

Once formulated, the sunscreen of the present invention may be included as one component of a zinc cream or of cosmetic products such as foundation, lipstick or tanning lotion. For the purposes of this discussion, however, we will be describing the use of the topical zinc sunscreen formulation for use in a sunscreen. This is not intended to limit the scope of the invention in any way.

Throughout the following illustrative examples, particular ingredients have been nominated by way of example only and are not intended to limit the scope of the present invention in any way. These examples are intended merely to show the best method of formulating a sunscreen or manufacturing the particles known to the applicant at the date of filing of the present application. It is expected that a person skilled in the art of The BET surface area was 38 m²/g which corresponds to a spherical particle size of 28 nm.

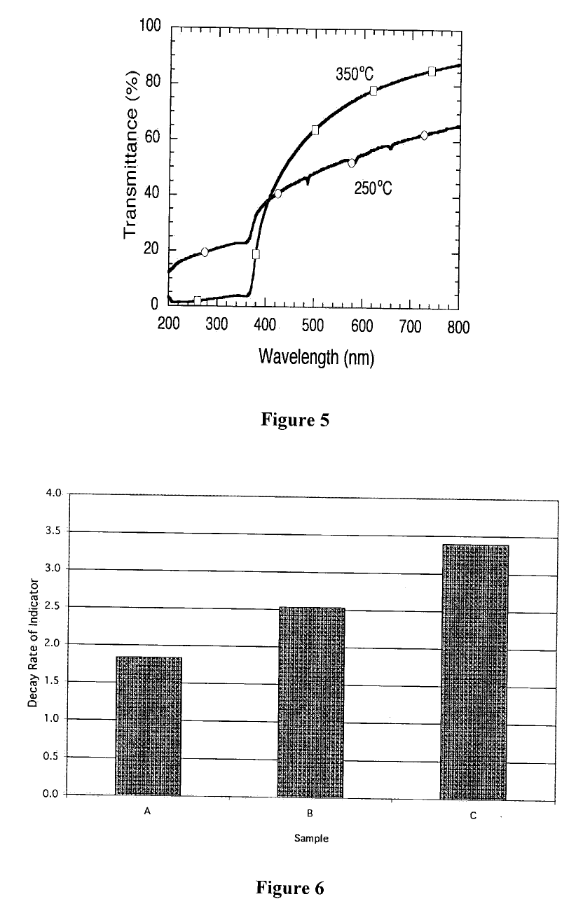

UV-Vis Spectroscopy measurement of a diluted slurry of the sample in deionised water having 0.01 wt % of ZnO and 0.0008 wt % of Dispex-A40 was carried out and compared with a sample heat treated at 350° C. following milling in accordance with Example 1. The aqueous suspension of the powder heat treated at 350° C. had a high transmittance in visible light range as well as high absorption in UV light range. On the other hand, the suspension of the powder heat-treated at 250° C. resulted in poor transmittance in visible light range and low absorption in UV light range (FIG. 5).

Heat treatment of the powder at 350° C. resulted in a slightly larger particle size in comparison to heat treatment at 250° C. On the other hand, as shown in FIG. 5 the UV-Vis measurements showed a significant decrease in the visible transparency and increase in UV transparency of the sample heat treated at 250° C. even though the particle size was smaller than that for the 350° C. sample. Without wishing to be bound by theory, it is believed that this result can be attributed to the particles heat treated at 250° C. having a higher reactivity, causing an increase in the effective particle size due to agglomeration. Particles heat-treated at 350° C. exhibited a significantly increased dispersibility associated with the effect of the higher heat treatment temperature stabilising the particle surfaces.

### EXAMPLE 3

### Photocatalytic Stability

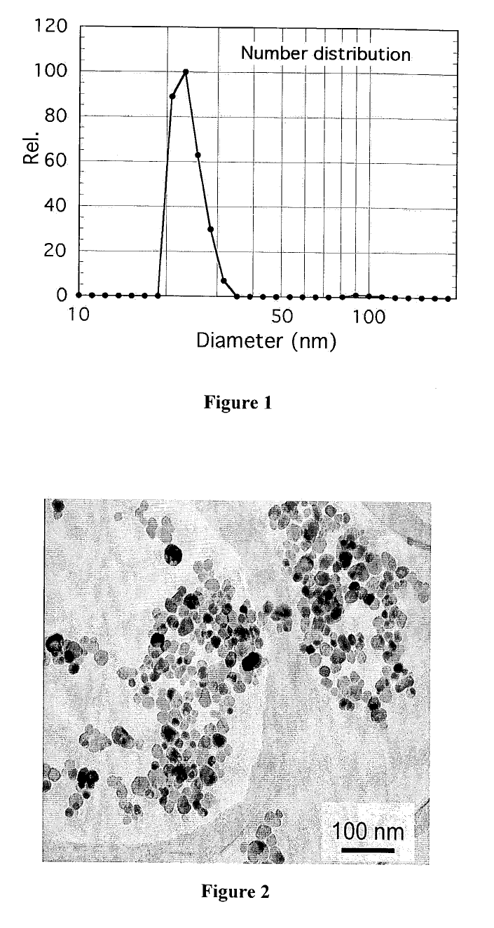

An aqueous slurry of mechano-chemically produced ZnO (sample A) was prepared following the method described in Example 1. Sample A had a BET surface area of 44.1 m²/g which corresponds to the spherical particle size of 24 nm. The ZnO particles were coated with stearic acid to form powder dispersed in Isostearyl Benzoate (C17 alkyl Benzoate), as described in Example 4. The suspension was diluted into 0.01 wt % in Isostearyl Benzoate, and ultrasonicated for 30 min. Commercially available dry ZnO powders synthesised by vapour condensation method (sample B) and wet chemical precipitation method (sample C), respectively, were The Photon Correlation Spectroscopy (PCS) measurements of the diluted slurry in deionised water having 0.01 wt % of ZnO and 0.0008 wt % of Dispex-A40 are shown in FIG. 1. The measurements showed that number-weighted mean particle size was 23.5 nm with standard deviation of 3.3 nm (14.%).

Examination of the slurry by transmission electron microscopy (TEM) (FIG. 2) showed that the particles are mostly single-crystalline particles having particle sizes of 10-50 nm.

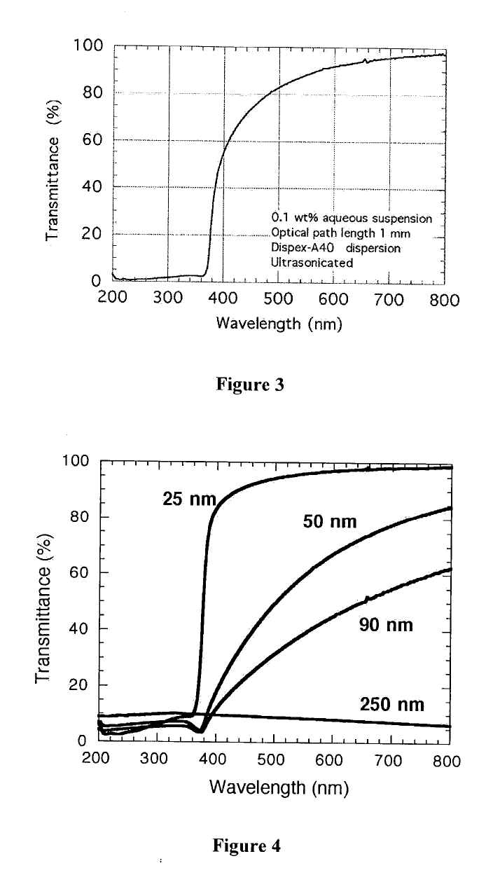

FIG. 3 shows UV-Vis Spectroscopy results for a diluted slurry of the ZnO in deionised water having 0.01 wt % of ZnO and 0.0008 wt % of Dispex-A40 dispersant. The measurements show that the suspension has a high transmittance in visible light range, over 80% at 500 nm and strong absorption in UV light range, indicative of fully dispersed 30 nm particles.

The UV-Vis Spectroscopy for the ZnO slurry was compared to slurries containing larger particle sizes. FIG. 4 shows a comparison of the results for the ZnO described above with slurries containing ZnO of 50 nm, 90 nm, and 250 nm mean particle size, respectively. All slurries were prepared in an identical manner using Dispex A40. The measurements show that the visible transmittance increases significantly with decreasing particle size, while the UV transmittance decreases with decreasing particle size.

### EXAMPLE 2

### Stabilisation of ZnO Particles by Heat Treatment

A sample milled in the same manner as in Example 1, was heat treated at 250° C. for 1 hour in air and cooled to room temperature. Examination of the heat-treated powders by X-ray diffraction showed that the phases present in the powder consisted of ZnO and NaCl.

Examination of the milled and heat treated sample using X-ray diffraction after removal of the NaCl and drying at 60° C. showed that the powder consisted of only the ZnO phase and the crystallite size estimated from the broadening of diffraction peaks was 18 nm. formulating sunscreens would understand that numerous variations of the specific quantities used and/or substitutions of the specific choice of components may be made without altering the essential characteristics of a sunscreen so formulated. All such variations are considered to be within the scope of the present invention for which the following examples are for illustrative purposes only.

### EXAMPLE 1

### Preparation of Nano-Sized ZnO Using Mechano-Chemical Processing

The raw materials used were anhydrous ZnCl₂ powder (Fluka, >98.0%, −10 mesh), Na₂CO₃ powder (Sigma, 99.8%, −100 mesh), and NaCl powder (Cleeze, 99.5%, −10 mesh). 5 kg of the starting powder mixture of ZnCl₂, Na₂CO₃ and NaCl in a molar ratio of 1:1:3.4 corresponding to the reaction

ZnCl₂+Na₂CO₃+3.4 NaCl [symbol] ZnO+5.4 NaCl+CO₂

was loaded into a 33 litre attrition mill, together with 100 kg of 5 mm hardened steel grinding balls. Mechanical milling was carried out for 90 min using an effective impeller tip speed of 4 m/s. The temperature within the mill during milling was approximately 75° C. Following milling, the powder was heat treated at 350° C. for 1 hour in air, and cooled to room temperature. Examination of the heat-treated powder by X-ray diffraction showed that the phases present in the powder consisted of ZnO and NaCl.

The milled and heat-treated powder was slurried into filtered deionised water to dissolve and remove the NaCl by-product/diluent phase. Using a settling and filtration technique, the salt content in the nanopowder-containing slurry was reduced to less than 10 ppm.

Examination of the slurry dried at 60° C. by X-ray diffraction showed that the powder consisted of only the ZnO phase. The crystallite size estimated from the broadening of the diffraction peaks was 26 nm. The surface area of the dried slurry measured by Brunauer-Emmett-Teller (BET) method was 40.9 m 2/g which corresponds to a spherical particle size of 26 nm. dispersed in water by ultrasonication, and coated with stearic acid to form powder dispersed in Isostearyl Benzoate, as described in Example 4. Samples B and C had BET surface areas of 13.1 and 13.8 m²/g respectively, which corresponds to spherical particle sizes of 82 and 77 nm, respectively. The suspension was diluted into 0.01 wt % in Isostearyl Benzoate, and ultrasonicated for 30 min.

100 g of 1,1-diphenyl-2-picrylhydrazyl (DPPH) radical solution (0.01 wt %) in Isostearyl Benzoate was made in a glass flask. The flask was wrapped with aluminium foil to prevent photo degradation.

2 g of the 0.01 wt % ZnO suspension, 4 g of 0.01 wt % DPPH solution, and 14 g of Isostearyl Benzoate were mixed in a glass beaker to make up 0.001 wt % of ZnO and 0.002 wt % of DPPH in Isostearyl Benzoate. The mixture was stirred in dark for 10 min. The mixtures of ZnO and DPPH in Isostearyl Benzoate for the Samples A, B and C were placed under mercury UV light. The mixture was constantly stirred using a magnetic stirrer.

UV-Vis spectra of the mixtures were measured at a wavelength range of 400-700 nm, before and during UV exposure at 5-min interval. Isostearyl Benzoate was used as a reference sample. The change in transmittance at 520 nm, which is the peak position of the absorption band of DPPH, as a function of UV exposure time was calculated, and defined as Decay Rate. DPPH is attacked by the photocatalytic activities of ZnO, resulting in the disappearance of its purple colour originated from the absorption band. Therefore, the decay rate is a measure of photocatalytic activities of ZnO.

Normally, high surface areas yields higher photocatalytic activities due to the larger reaction interface. However, surprisingly, the decay rate and hence photocatalytic activity for the mechano-chemically produced ZnO (sample A) was significantly lower than that of samples B and C, as shown in FIG. 6.

transparency and low values of UV transparency indicative of well-dispersed nano size ZnO particles in the canola oil.

### EXAMPLE 6

### Method for Making Dry Re-Dispersible ZnO

A slurry of 30 nm ZnO in water of total mass 853 grams (11.2 wt % ZnO) was prepared and added to a solution of 14.6 grams of stearic acid dissolved in 97.3 grams of hexane. The liquids were mixed together in a Hobart planetary mixer for 1 hour during which time the zinc oxide was transferred from the water to the hexane phase. The water was removed and 4.9 grams of Solsperse 3000 dispersant and 100 grams of hexane were added and the zinc oxide was fully dispersed using a high shear mixer. The hexane was removed by evaporation at 60° C. for 3 hours resulting in a dry, free flowing powder.

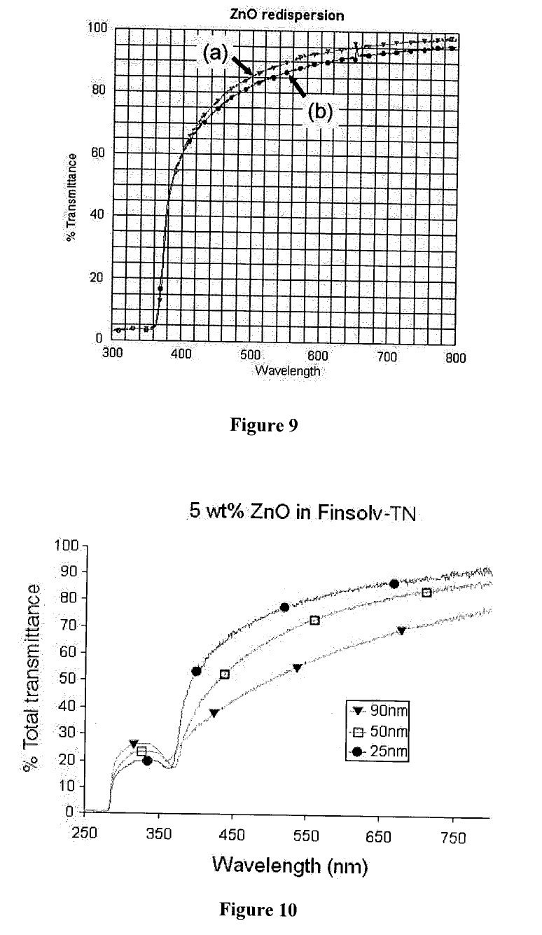

The dry ZnO powder was then dispersed in hexane to form a 0.01 wt % solution using an ultrasonic bath. UV-Vis spectroscopy measurements carried out on the sample prior to drying and the sample after drying and redispersion in a 10 mm pathlength sample holder are shown in FIG. 9. The visible specular transmittance curve for the dried and redispersed sample is nearly identical with the undried sample, indicating that it was possible to fully redisperse the dried powder.

### EXAMPLE 7

### Properties of Sunscreen Formulations

To demonstrate the enhanced properties of sunscreens incorporating the ZnO particles according to at least a preferred embodiment of the present invention, UV-Vis measurements were carried out on samples prepared by dispersing ZnO particles manufactured using the method of Example 1 into Isostearyl Benzoate using the method of Example 4. Isostearyl Benzoate is a common base used for sunscreen formulations. The concentration of ZnO was varied from 2 wt % to 30 wt %. For comparison, samples containing 50 nm, 90 nm and 250 nm mean particle size dispersed in Isostearyl Benzoate were also tested.

FIG. 10 shows UV-Vis curves for samples with mean particle sizes of 25, 50 and 90 nm. It is seen that the total transmittance in the visible light region from 400 to 700 nm increases with decreasing particle size, while in the UV region (200-400 nm) the

### EXAMPLE 4

### Method for Coating ZnO with Stearic Acid and Transferring to Isostearyl Benzoate

A slurry of 30 nm ZnO in water of total mass 1.63 kg (11.2 wt % ZnO) was prepared. The ZnO was manufactured according to Example 1. Separately, 27 grams of stearic acid (corresponding to 15% of the mass of ZnO) was mixed with 180 grams of Isostearyl Benzoate until the stearic acid dissolved.

Both phases were then loaded into a Hobart planetary mixer and mixed for 2 hours. During mixing the ZnO was transferred from the water phase into the Isostearyl Benzoate phase, forming a thick paste. The water was then removed.

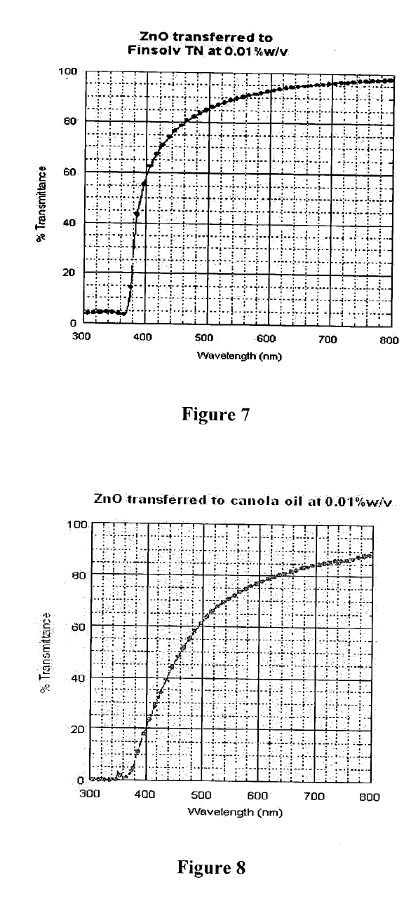

The mixture of Isostearyl Benzoate and ZnO was diluted to 30 wt % ZnO and 9 grams of Solsperse 3000 (5 wt % relative to ZnO) was added. The sample was dispersed using an ultrasonic disperser. The sample was further diluted to 0.01% w/v ZnO in Isostearyl Benzoate. UV-visible specular transmittance measurements carried out in a 10 mm pathlength sample holder are shown in FIG. 7. The UV-Vis measurements showed high values of the visible transparency and low values of UV transparency indicative of well-dispersed nano size ZnO particles in the Isostearyl Benzoate.

### Example 5

### Method for Coating ZnO with Stearic Acid and Transferring to Canola Oil

A slurry of 30 nm ZnO in water of total mass 1.63 kg (11.2 wt % ZnO) was prepared. The ZnO was manufactured according to Example 1. Separately, 38 grams of stearic acid (corresponding to 15% of the mass of ZnO) was mixed with 251 grams of canola oil until the stearic acid dissolved.

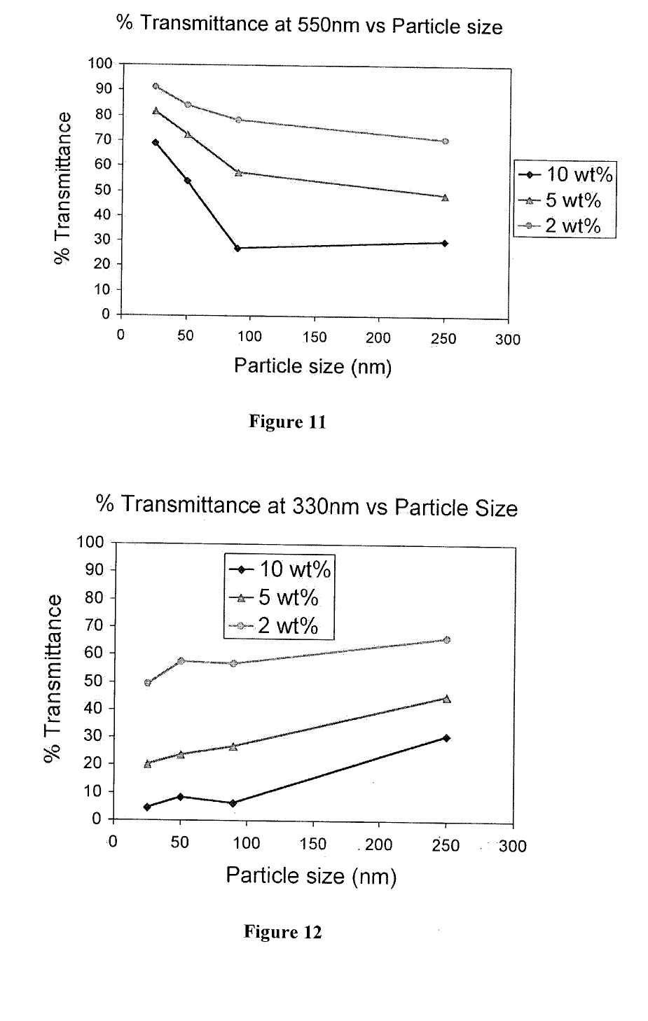

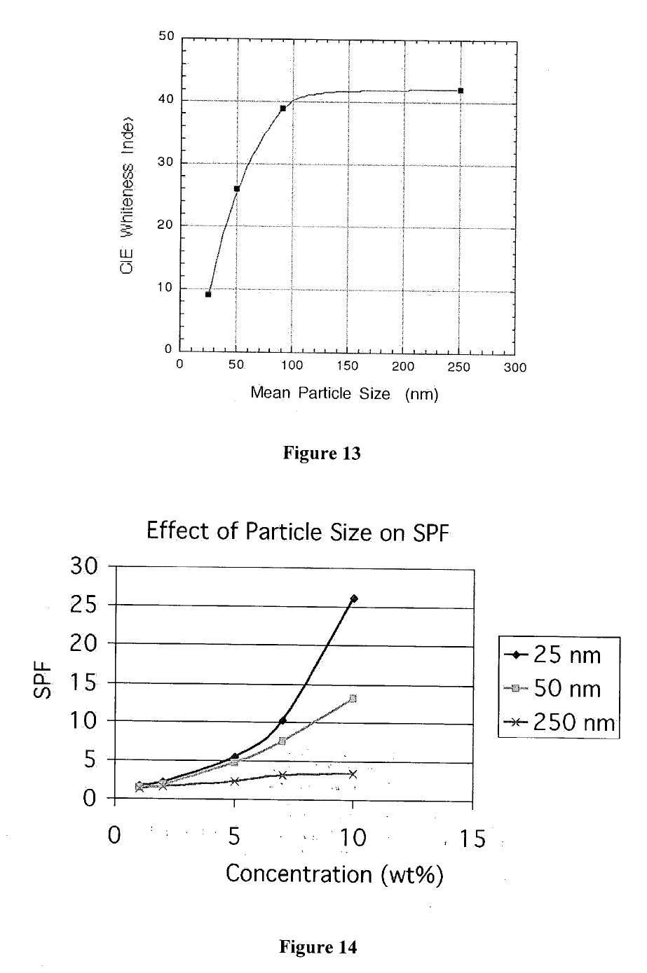

Both phases were then loaded into a Hobart planetary mixer and mixed for 2 hours. During mixing the ZnO was transferred from the water phase into the canola oil phase, forming a thick paste. The water was then removed. The sample was dispersed using an ultrasonic disperser and further diluted to 0.01% w/v ZnO in canola oil. UV-visible transmittance measurements carried in a 10 mm pathlength sample holder are shown in FIG. 8. The UV-Vis specular measurements showed high values of the visible transmittance decreases (absorptance increases) with decreasing particle size. FIG. 11 shows the variation of visible transmittance at 550 nm with particle size. FIG. 12 shows the variation of UV transmittance at 330 nm with particle size. FIG. 13 shows the effect of the mean particle size on the CIE Whiteness Index. FIG. 14 shows the effect of particle size and concentration on the in-vitro SPF. It is seen that for a given concentration of ZnO, the highest SPF is achieved with the smallest particle size. On the basis of FIGS. 10-14 it is concluded that significant enhancement of sunscreen performance is achieved by decreasing the mean particle size of ZnO to below 30 nm.

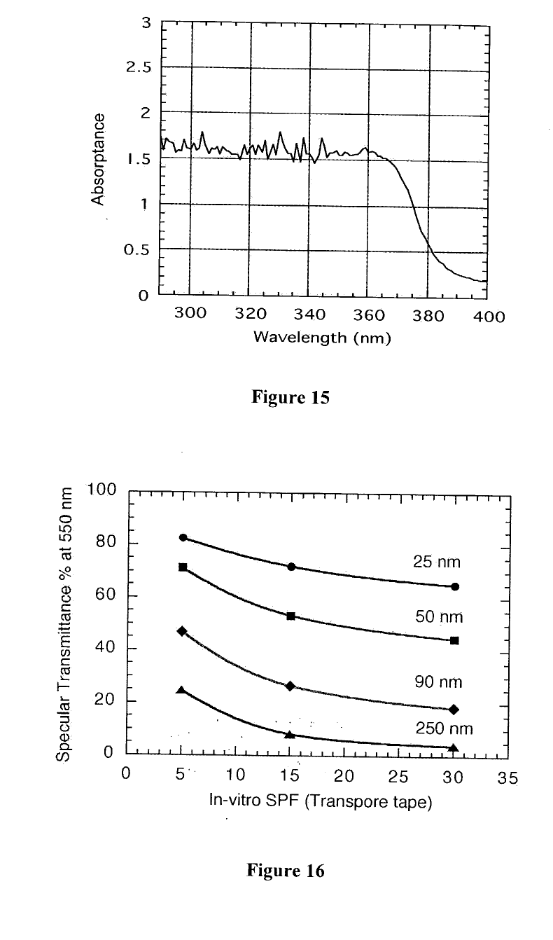

FIG. 15 shows measurements of absorptance as a function of wavelength in the UV region for a sample containing 30 wt % ZnO dispersed in Isostearyl Benzoate. The UV measurements were made using the Transpore tape method. Sunscreen performance values for this sample are shown in Table 1.

| Property | Value |
|---|---|
| In Vitro SPF | 31 |
| UVA/UVB | 0.73 |
| Critical Wavelength | 371 nm |
| Diffuse reflectance | 13.7% |
| CIE Whiteness | 42.5 |

Table 1: Sunscreen performance values for 30 wt % ZnO in Isostearyl Benzoate

FIG. 16 shows the effect of particle size on transparency as a function of in-vitro SPF (Transpore tape method). This figure is drawn using FIGS. 11 and 14 in the patent. FIG. 11 is a relation between size, wt % and transmittance. FIG. 14 is a relation between size, wt % and in-vitro SPF. Therefore, from FIGS. 11 and 14, the relation between size, transmittance and in-vitro SPF can be deduced via the wt % of ZnO.

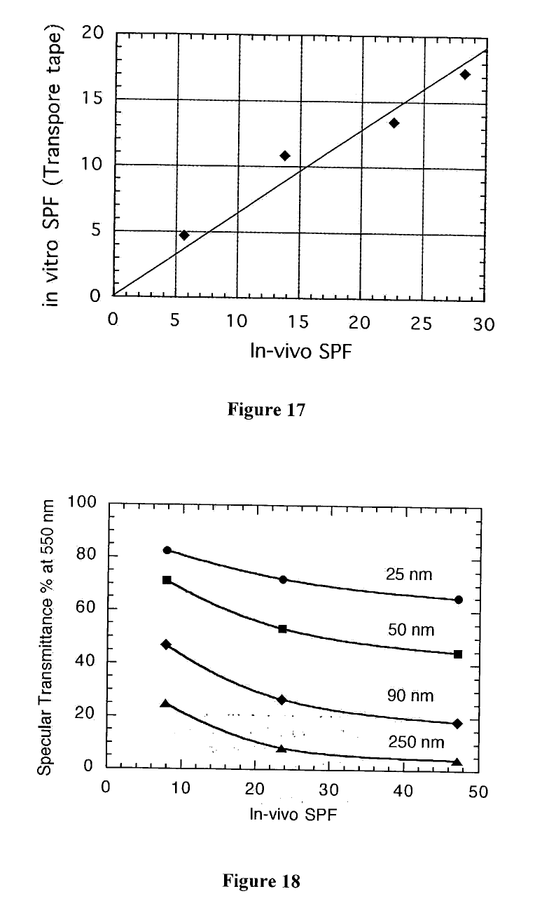

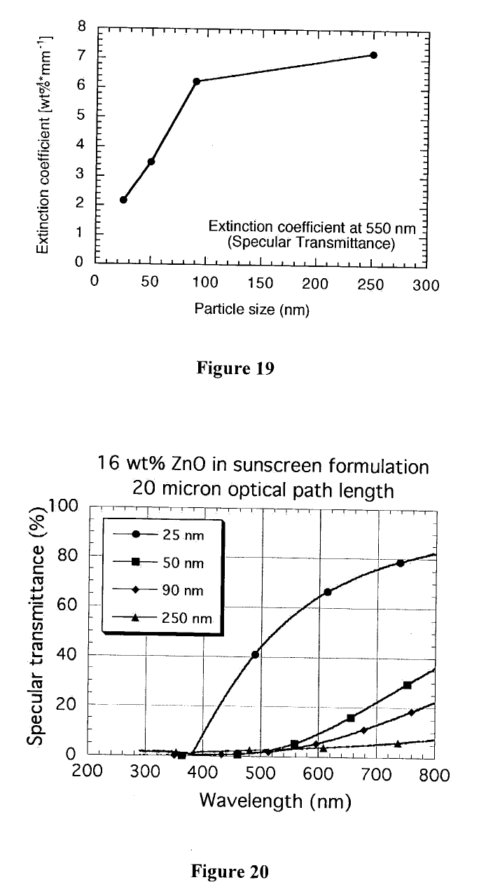

FIG. 16 shows the importance of small particle size for transparent sunscreen having a high SPF value; smaller particles contributing to higher transparency at a fixed SPF. Of particular importance is the increase in Transmittance % as the particle size is reduced from 50 nm to 25 nm. Since there is a linear correlation between in-vitro SPF (Transpore Tape method) and in-vivo SPF as shown in FIG. 17, FIG. 16 can be re-drawn as Transmittance % as a function of in-vivo SPF (FIG. 18). FIG. 19 shows the effect of particle size on the specular extinction coefficient, α, defined as Transmittance %=100* exp (−α*C*L), where C is the concentration [wt %] and L is the optical path length [mm]. The rapid decrease in α below 100 nm is of great significance to the transparency of sunscreens.

FIG. 19 can be deduced from FIG. 11 where Transmittance % was plotted against particle size and concentration. It is to be noted that the specular extinction coefficient is, by its definition, normalised with concentration and film thickness. As such, it is a direct measure of scattering power of particles having different particle sizes. This means that, in order to obtain high transparency at a high SPF value, it is essential to use small particles having a low specular extinction coefficient.

### EXAMPLE 8

### Effect of ZnO Particle Size on the Properties of Water-in-Oil Emulsion of Chemical Free Sunscreen

UV-Vis measurements were carried out on samples prepared by using the following formulation. This formulation contains no organic/chemical UV screening agent.

| Ingredients: | % w/w |
|---|---|
| Water | 35.75 |
| Propylene glycol | 3 |
| Magnesium sulphate | 2 |
| Keltrol HF | 0.15 |
| Zinc oxide | 16 |
| Finsolv-TN | 38 |
| Arlacel P135 | 3 |
| Monomuls 90-018 | 1 |
| Performalene 400 | 0.7 |
| Liquid Germall Plus | 0.4 |

The mean particle sizes of the zinc oxide powders for use in the above formulation were 25 nm (using the manufacturing method described in Example 1); 50 nm; 90 nm; and 250 nm.

The first step in the preparation of this formulation was to prepare a water phase by dissolving magnesium sulphate and propylene glycol in water. Keltrol was then dispersed in the water phase by adding it slowly while stirring at 80-85° C. An oil phase was prepared by heating Zinc Oxide in Finsolv-TN along with Arlacel P135, Monomuls 90-018 and Performalene 400 to 90-95° C. for 5 minutes. The mixture was stirred until melted. The water phase was then added to the oil phase. The mixture was stirred using a high shear mixer, and then cooled down to 40-45° C. Germall Plus was then mixed in.

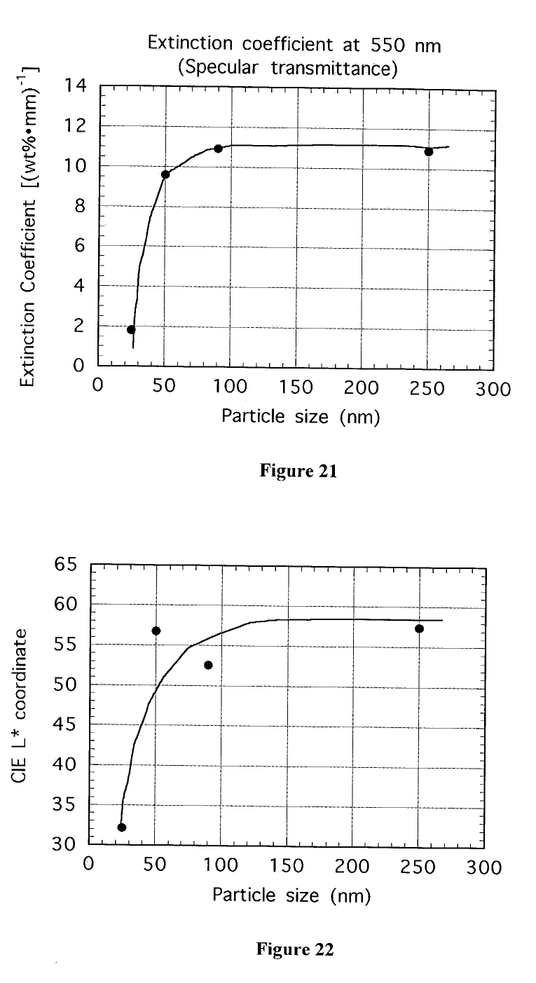

FIG. 20 shows the specular transmittance of de-emulsified sunscreens using a 20 micron quartz cell. FIG. 21 shows the specular extinction coefficient at 550 nm calculated from FIG. 20. FIG. 22 shows CIE L* coordinate calculated from diffuse reflectance measurements. CIE L* coordinate is a measure of brightness of samples, and thus an indication of whitening effect.

From FIGS. 20, 21 and 22, it is evident that the smaller the mean particle size of the zinc oxide particles the better the clarity achieved. There was a surprising improvement in the transparency for a mean particle size of 25 nm than would have been extrapolated from the data for 50 nm particles or greater mean particle diameter.

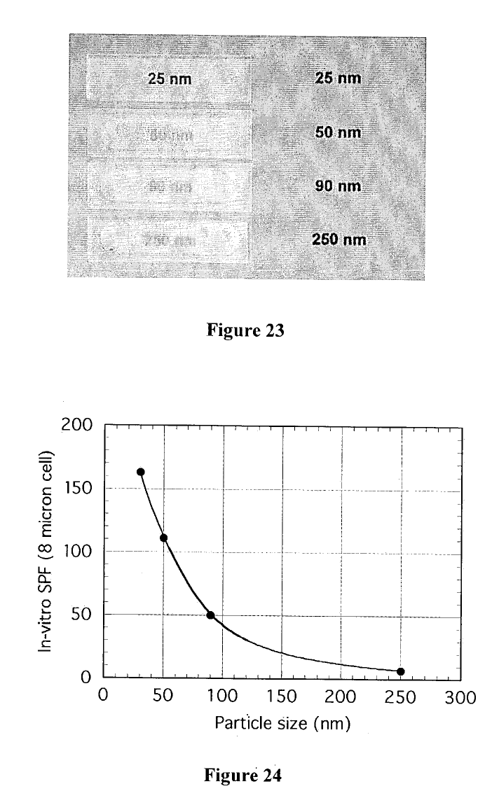

FIG. 23 illustrates the superior transparency of the 25 nm zinc oxide formulation using images of the sunscreen formulations in an 8-micron optical-path-length quartz cell. The mean particle size for each formulation was printed on each of two columns using the same font/font-size. The quartz cell was placed on top of the letters that correspond to the particle size in the sunscreen formulation. It is clearly evident that the sample containing zinc oxide particles with a mean particle size of 25 nm had the highest transparency of the formulations so tested.

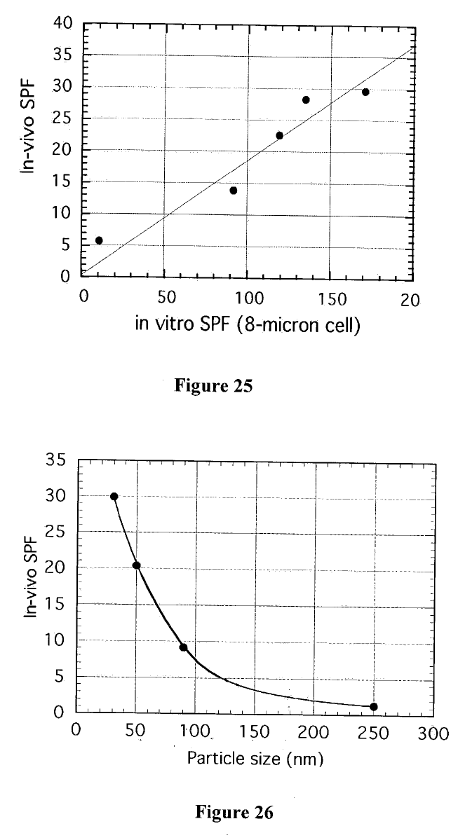

Using a quartz cell having an optical-path-length of 8 micron, in-vitro SPF measurements were carried out for each of the four formulations having a mean particle size of 25 nm, 50 nm, 90 nm and 250 nm. FIG. 24 shows the in-vitro SPF as a function of particle size. The SPF value was higher for smaller particles. Since the in-vitro SPF measured using 8 micron cell has a linear relation with in-vivo SPF values for ZnO suspension in Finsolv-TN (FIG. 25), FIG. 20 can be re-plotted as in FIG. 26.

### EXAMPLE 9

### Water-in-Oil Emulsion of Chemical Free Sunscreen Having a SPF Value Greater than 30, and Excellent Clarity on the Skin

UV-Vis measurements were carried out on samples prepared by using the following formulation. This formulation contains no organic/chemical UV screening agent.

| Ingredients: | % w/w |
|---|---|
| Water | 42.65 |
| Propylene glycol | 3 |
| Magnesium sulphate | 2 |
| Keltrol HF | 0.15 |
| Zinc oxide nano-particles | 16.77 |
| Finsolv-TN | 26.23 |
| Isopropyl palmitate | 2 |
| Dehymuls PGPH | 1 |
| Monomuls 90-018 | 1 |
| Zinc stearate | 1 |
| Beeswax | 2 |
| Liquid Germall Plus | 0.2 |

As a first step in preparing this formulation, a water phase was made by dissolving magnesium sulphate and propylene glycol in water. Keltrol was dispersed in water phase by adding it slowly while stirring at 80-85° C. An oil phase was then prepared by heating Zinc Oxide in Finsolv-TN along with Isopropyl Palmitate, beeswax, Dehymuls and Monomuls to 80-85° C. for 5 minutes. The mixture was stirred until melted. The water phase was then added to the oil phase and the mixture was stirred using a high shear mixer, and then cooled down to 40-45° C. Germall Plus was then mixed in.

In-vivo SPF test and in-vitro UVVis measurements were conducted on this formulation with the results being presented in Table 2 below:

TABLE 2
Sunscreen performance values for 16.77 wt % ZnO in the
chemical-free sunscreen formulation.

| Property | Value |
|---|---|
| In-vivo SPF | 30.2 |
| UVA/UVB | 0.77 |
| Critical wavelength | 370 nm |
| Extinction coefficient [wt % mm]⁻¹ for | 1.50 |
| specular transmittance at 550 nm | |
| CIE L* coordinate (20 micron thick film) | 29.6 |

### EXAMPLE 10

### Water-in-Oil Emulsion of Sunscreen Including Organic UV Absorbers having a SPF Value of 30+, and Excellent Clarity on the Skin

UVVis measurements were carried out on samples prepared by using the following formulation containing no organic/chemical UV screening agents.

| Ingredients: | % w/w |
|---|---|
| Water | 42.65 |
| Propylene glycol | 3 |
| Magnesium sulphate | 2 |
| Keltrol HF | 0.15 |
| C₁₂₋₁₅ alkyl benzoates | 24 |
| Zinc oxide nano-particles | 6 |
| Parsol MCX (organic UV blocker) | 8 |
| Parsol 5000 (organic UV blocker) | 8 |
| Isopropyl palmitate | 8 |
| Dehymuls PGPH | 3 |
| Monomuls 90-0 18 | 1 |
| BHT | 0.05 |
| PCL Liquid | 4.95 |
| Zinc stearate | 2 |
| Beeswax | 2 |
| Cab-O-Sil TS 530 | 1 |
| Germall Plus | 0.2 |

A water phase was prepared by dissolving magnesium sulphate in water. Keltrol was dispersed in the water phase by adding it slowly while stirring at 80-85° C. An oil phase was prepared by heating zinc oxide in C12-15 alkyl benzoates along with Isopropyl palmitate, PCL Liquid and zinc stearate to 80-85° C. for 5 min. Beeswax, Dehymuls and Monomuls were then added and the mixture was stirred until melted. Cab-O-Sil, Parsol MCX, Parsol 5000, BHT, propylene glycol were then added and stirred for 2-3 min. The water phase was then added to the oil phase. The mixture was stirred using a high shear mixer, and then cooled down to 40-45° C. Germall Plus was then mixed in.

In-vivo SPF test and in-vitro UVVis measurements have been carried out. Sunscreen performance values for this sample are shown in Table 3.

TABLE 3
Sunscreen performance values for 6.0 wt % ZnO in the
chemical-free sunscreen formulation.

| Property | Value |
|---|---|
| In-vivo SPF | 41.2 |
| UVA/UVB | 0.39 |
| Critical wavelength | 363 nm |
| Extinction coefficient [wt % mm]⁻¹ for | 1.83 |
| specular transmittance at 550 nm | |
| CIE L* coordinate (20 micron thick film) | 22.1 |

### EXAMPLE 11

### Extinction Coefficient for Specular Transmittance of 550 nm for Various Formulations

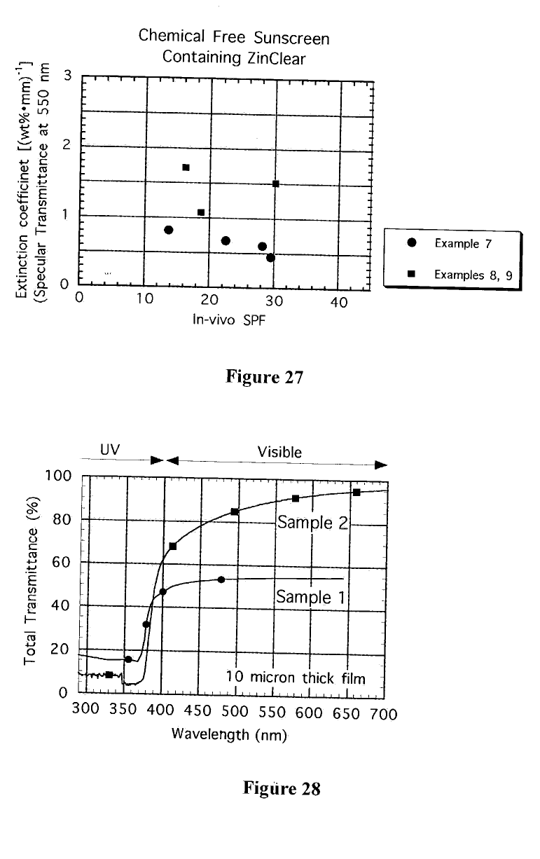

FIG. 27 illustrates the extinction coefficient at a specular transmittance of 550 nm for zinc oxide particles with a mean particle size of 25 nm dispersed in Finsolv-TN as per Example 7, as well as the formulations of Examples 7, 8 and 9. From FIG. 27 it is readily apparent that the values of the specular extinction coefficient for either complex formulations such as those of Examples 7, 8, and 9 as well as a simple formulation using Finsolv-TN alone are less than 2.0 (wt %.mm)⁻¹ with the simple formulations being less than 1.0 (wt %.mm)⁻¹.

### EXAMPLE 12

### Comparison with Other Commercially Available Products

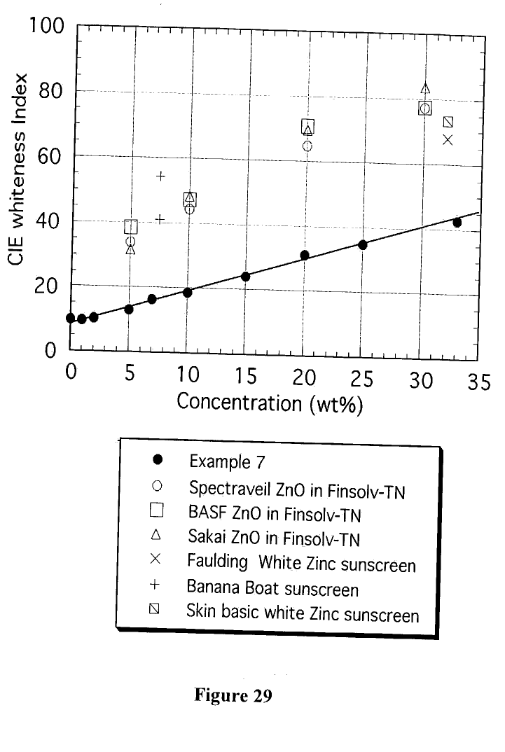

FIG. 28 compares the UV-Vis total transmittance spectra of two samples, designated Sample 1 and Sample 2 for 10 micron thick films. The data presented for Sample 1 was calculated from the data disclosed in an Affidavit filed under 37 C.F.R. 1,132 by Mark Mitchnick during re-examination of U.S. Pat. No. 5,587,148 for a dispersion of 122 nm ZnO in mineral oil (40 wt %, 28 micron thick film), using Beer's law. Sample 2 was a dispersion of 25 nm sized ZnO particles in accordance with the present invention in Finsolv-TN oil (40 wt %, 28 micron film thick). The UV-Vis data for Sample 2 was calculated from the spectrum of Sample 2, using Beer's law.

FIG. 28 demonstrates that Sample 2 has a significantly increased transparency in the visible light range compared with Sample 1. Also the UV screening efficiency significantly increased for Sample 2 over Sample 1, which corresponds to approximately a factor of two increase in SPF value.

The total transmittance is a sum of the diffuse and specular transmittance values. Specular transmittance is directly related to transparency. For example, “clarity” of plastic sheets is defined as specular transmittance according to ASTM D 1746-97. Diffuse transmittance is caused by light scattering by particles, and thus a measure of cloudiness or whitening effect. If the whitening effect is large, diffuse transmittance is large as well, and hence total transmittance may increase, in spite of low transparency. It can be misleading to use total transmittance for the evaluation of transparency. Therefore, transparency should be evaluated using specular transmittance measurements, as explained below.

In FIG. 28, values of total transmittance of Samples 1 and 2 are compared for the argument of transparency, only because the Affidavit filed under 37 C.F.R. 1,132 by Mark Mitchnick during re-examination of U.S. Pat. No. 5,587,148 discloses total transmittance spectra but not specular transmittance spectra.

FIG. 29 shows a comparison of the whiteness index of various commercially available formulations with that of the present invention when the mean particle size of the zinc oxide particles is 25 nm. The whiteness index is shown as a function of the concentration of such zinc oxide powders within the composition and a linear relationship between whiteness index and weight percent of zinc oxide was observed for the formulation made in accordance with the present invention. More importantly, FIG. 29 clearly indicates the that the sunscreen compositions of the present invention provide significantly reduced whiteness over the full range of weight percentages of zinc oxide particles likely to be included in such a sunscreen when compared with other commercially available sunscreens.

The sunscreen composition of the present invention and the zinc oxide particles included therein have many advantages over the prior art including but not limited to the following:

(i) the ability for the first time to make available to the market a sunscreen that can deliver a dermatologically acceptable level of SPF in a substantially visibly clear, transparent sunscreen without needing to include chemical UV sunscreen agents

(ii) the ability due to (i) to protect the user from the potentially unfavourable effects of chemical UV screening agents

(iii) a more photostable sunscreen due to the ability to avoid the inclusion of chemical UV screening agents.

(iv) improved cosmetic acceptability due to far superior whiteness and specular extinction coefficients compared with prior art sunscreens without a reduction in SPF rating of a sunscreen composition;

(v) improved UV radiation attenuation and increased visible transparency;

(vi) improved dispersibility;

(vii) the ability to supply the particles in the form of a dry re-dispersible powder to reduce the cost of transportation of the powders to formulators.

(viii) a high reproducibility and control of the particle size, size distribution and agglomeration using mechano-chemical processing with suitable heat treatment and optional use of particle coatings It will be apparent to persons skilled in the materials engineering and sunscreen formulation arts that numerous enhancements and modifications can be made to the above-described powders, method of production of the powders and sunscreen compositions without departing from the basic inventive concepts. For example, similar results may be obtained using titanium dioxide or a mixture of zinc oxide and titanium dioxide as the physical UV sunscreen agent. All such modifications and enhancements are considered to be within the scope of the present invention, the nature of which is to be determined from the foregoing description. Furthermore, the preceding examples are provided for illustrative purposes only, and are not intended to limit the scope of the invention.

1. A substantially visibly clear and transparent topical sunscreen composition for shielding the skin from ultraviolet radiation, said composition comprising:
a sufficient weight percentage of nano-sized particles of a physical UV screening agent to provide said dermatologically acceptable level of SPF and broad spectrum protection from UVA and UVB radiation in a dermatologically acceptable carrier;
whereby said composition contains no chemical UV screening agents.

2. A substantially visibly clear and transparent topical sunscreen composition according to claim 1 wherein the dermatologically acceptable level of SPF is greater than 8+.

3. A substantially visibly clear and transparent topical sunscreen composition according to claim 1 wherein the dermatologically acceptable level of SPF is greater than 15+.

4. A substantially visibly clear and transparent topical sunscreen composition according to claim 1 wherein the dermatologically acceptable level of SPF is greater than 30+.

5. A substantially visibly clear and transparent topical sunscreen composition according to claim 1 wherein the physical UV screening agent is zinc oxide.

6. A substantially visibly clear and transparent topical sunscreen composition according to claim 1 wherein the physical UV screening agent is zinc oxide with up to 10% of one or more titanium dioxide or other physical UV screening agents.

7. A substantially visibly clear and transparent topical sunscreen composition according to claim 1 wherein said composition has a specular extinction coefficient of less than 2 (wt % mm)⁻¹ measured at a wavelength of 550 nm.

8. A substantially visibly clear and transparent topical sunscreen composition according to claim 1 wherein said composition has a specular extinction coefficient of less than 1 (wt % mm)⁻¹ measured at a wavelength of 550 nm.

9. A substantially visibly clear and transparent topical sunscreen composition according to claim 1 wherein said physical UV screening agent has a mean particle size of less than 30 nm and a narrow particle size distribution characterised in that, based on a number-weighted size distribution measured by photo-correlation spectroscopy, the number-weighted size distribution has a standard deviation of less than 20 nm.

10. A substantially visibly clear and transparent topical sunscreen composition according to claim 9 wherein the number-weighted size distribution measured by photo-correlation spectroscopy has a standard deviation of less than 10 nm.

11. A substantially visibly clear and transparent topical sunscreen composition according to claim 9 wherein the number-weighted size distribution measured by photo-correlation spectroscopy has a standard deviation of less than 5 nm.

12. A substantially visibly clear and transparent topical sunscreen composition according to claim 1 wherein the particles have a photoactivity which is reduced by treatment with a surfactant.

13. A substantially visibly clear and transparent topical sunscreen composition according to claim 9 wherein the surfactant is a steric surfactant.

14. A substantially visibly clear and transparent topical sunscreen composition according to claim 1 wherein the particles are coated with a layer of one or more of a metal hydroxide, a metal oxide or a hydrous metal oxide.
